SEQUENCE LISTING (69 sequences)

SEQ ID NO: 1 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLVFLCLQYRLRGKLRAEIENLHRTFDPHFLRVPCWKITLFVIVPVLGPLVALIICYNWLHRRLAGQFLEELRNPF

SEQ ID NO: 2 - protein, 247 aa, Mus musculus
MACLWSFSLPSCFLSLLLLLLLQLSCSYAGQFRVIGPGYPIRALVGDEAELPCRISPGKNATGMEVGWYRSPFSRVVHLYRNGKDQDAEQAPEYRGRTELLKETISEGKVTLRIQNVRFSDEGGYTCFFRDHSYQEEAAMELKVEDPFYWVNPGVLTLIALVPTILLQVSVGLVFLFLQHRLRGKLRAEVENLHRTFDPHFLRVPCWKITLFVIVPVLGPLVALIICYNWLHRRLAGQFLEELRNPL

SEQ ID NO: 3 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 4 - protein, 154 aa, Mus musculus
MASQKRPSQRSKYLATASTMDHARHGFLPRHRDTGILDSIGRFFSGDRGAPKRGSGKVPWLKQSRSPLPSHARSRPGLCHMYKDSHTRTTHYGSLPQKSQHGRTQDENPVVHFFKNIVTPRTPPPSQGKGRGLSLSRFSWGGRDSRSGSPMARR

SEQ ID NO: 5 - protein, 626 aa, Homo sapiens
MIFLTALPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGPVGAVVAFAILIAIVCYITQTRRKKNVTESPSFSAGDNPPVLFSSDFRISGAPEKYESERRLGSERRLLGLRGEPPELDLSYSHSDLGKRPTKDSYTLTEELAEYAEIRVK

SEQ ID NO: 6 - protein, 626 aa, Mus musculus
MIFLATLPLFWIMISASRGGHWGAWMPSTISAFEGTCVSIPCRFDFPDELRPAVVHGVWYFNSPYPKNYPPVVFKSRTQVVHESFQGRSRLLGDLGLRNCTLLLSTLSPELGGKYYFRGDLGGYNQYTFSEHSVLDIVNTPNIVVPPEVVAGTEVEVSCMVPDNCPELRPELSWLGHEGLGEPTVLGRLREDEGTWVQVSLLHFVPTREANGHRLGCQAAFPNTTLQFEGYASLDVKYPPVIVEMNSSVEAIEGSHVSLLCGADSNPPPLLTWMRDGMVLREAVAKSLYLDLEEVTPGEDGVYACLAENAYGQDNRTVELSVMYAPWKPTVNGTVVAVEGETVSILCSTQSNPDPILTIFKEKQILATVIYESQLQLELPAVTPEDDGEYWCVAENQYGQRATAFNLSVEFAPIILLESHCAAARDTVQCLCVVKSNPEPSVAFELPSRNVTVNETEREFVYSERSGLLLTSILTIRGQAQAPPRVICTSRNLYGTQSLELPFQGAHRLMWAKIGPVGAVVAFAILIAIVCYITQTRRKKNVTESSSFSGGDNPHVLYSPEFRISGAPDKYESEKRLGSERRLLGLRGESPELDLSYSHSDLGKRPTKDSYTLTEELAEYAEIRVK

SEQ ID NO: 7 - protein, 92 aa, Homo sapiens
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 8 - protein, 92 aa, Mus musculus
MSELEKAMVALIDVFHQYSGREGDKHKLKKSELKELINNELSHFLEEIKEQEVVDKVMETLDEDGDGECDFQEFMAFVAMVTTACHEFFEHE

SEQ ID NO: 9 - protein, 277 aa, Homo sapiens [sequence omitted]

SEQ ID NO: 10 - protein, 277 aa, Mus musculus
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 11 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

SEQ ID NO: 12 - protein, 175 aa, Mouse
MDIAIHHPWIRRPFFPFHSPSRLFDQFFGEHLLESDLFSTATSLSPFYLRPPSFLRAPSWIDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVAAAPKK

SEQ ID NO: 13 - protein, 21 aa, Artificial sequence
  peptide
MEVGWYRSPFSRVVHLYRNGK

SEQ ID NO: 14 - protein, 21 aa, Artificial sequence
  peptide
MEVGWYRPPFSRVVHLYRNGK

SEQ ID NO: 15 - protein, 9 aa, Artificial Sequence
  peptide
SLLLELEEV

SEQ ID NO: 16 - protein, 9 aa, Artificial sequence
  peptide
SLYLDLEEV

SEQ ID NO: 17 - protein, 9 aa, Artificial sequence
  peptide
LMWAKIGPV

SEQ ID NO: 18 - protein, 9 aa, Artificial sequence
  peptide
VLFSSDFRI

SEQ ID NO: 19 - protein, 9 aa, Artificial sequence
  peptide
VLYSPEFRI

SEQ ID NO: 20 - protein, 9 aa, Artificial sequence
  peptide
SLSRFSWGA

SEQ ID NO: 21 - protein, 9 aa, Artificial sequence
  peptide
SLSRFSWGG

SEQ ID NO: 22 - protein, 9 aa, Artificial sequence
  peptide
KVEDPFYWV

SEQ ID NO: 23 - protein, 10 aa, Artificial sequence
  peptide
RTFDPHFLRV

SEQ ID NO: 24 - protein, 9 aa, Artificial sequence
  peptide
FLRVPCWKI

SEQ ID NO: 25 - protein, 10 aa, Artificial sequence
  peptide
KITLFVIVPV

SEQ ID NO: 26 - protein, 9 aa, Artificial sequence
  peptide
VLGPLVALI

SEQ ID NO: 27 - protein, 9 aa, Artificial sequence
  peptide
TLFVIVPVL

SEQ ID NO: 28 - protein, 10 aa, Artificial sequence
  peptide
RLAGQFLEEL

SEQ ID NO: 29 - protein, 9 aa, Artificial sequence
  peptide
FLYGALLLA

SEQ ID NO: 30 - protein, 14 aa, Artificial sequence
  peptide
HPIRALVGDEVELP

SEQ ID NO: 31 - protein, 20 aa, Artificial sequence
  peptide
VGWYRPPFSRVVHLYRNGKD

SEQ ID NO: 32 - protein, 26 aa, Artificial sequence
  peptide
LKVEDPFYWVSPGVLVLLAVLPVLLL

SEQ ID NO: 33 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 34 - protein, 457 aa, Homo sapiens [sequence omitted]

SEQ ID NO: 35 - protein, 419 aa, Homo sapiens
LKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 36 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 37 - protein, 110 aa, Homo sapiens; identical to SEQ ID NO: 33

SEQ ID NO: 38 - protein, 11 aa, Artificial sequence
  peptide
MRLLPLLALLA

SEQ ID NO: 39 - protein, 15 aa, Artificial Sequence
  peptide
SHLVEALYLVCGERG

SEQ ID NO: 40 - protein, 9 aa, Artificial sequence
  peptide
LYLVCGERG

SEQ ID NO: 41 - protein, 15 aa, Artificial sequence
  peptide
GIVEQCCTSICSLYQ

SEQ ID NO: 42 - protein, 5 aa, Artificial sequence
  peptide
XXRAA

SEQ ID NO: 43 - protein, 8 aa, Artificial sequence
  peptide
AGERGPPG

SEQ ID NO: 44 - protein, 15 aa, Artificial sequence
  peptide
AGGFDEKAGGAQLGV

SEQ ID NO: 45 - protein, 11 aa, Artificial sequence
  peptide
VGPAGGPGFPG

SEQ ID NO: 46 - protein, 1937 aa, Homo sapiens [sequence omitted]

SEQ ID NO: 47 - protein, 30 aa, Artificial sequence
  peptide
XLKLMATLFSTYASADTGDSGKGKGGKKKG

SEQ ID NO: 48 - protein, 14 aa, Artificial sequence
  peptide
GQFIDSGKAGAEKL

SEQ ID NO: 49 - protein, 14 aa, Artificial sequence
  peptide
DECSELKKDIDDLE

SEQ ID NO: 50 - protein, 12 aa, Artificial sequence
  peptide
NIFEXQVDAQPL

SEQ ID NO: 51 - protein, 22 aa, Artificial sequence
  peptide
YSLEHSTDDXASFSRALENATR

SEQ ID NO: 52 - protein, 20 aa, Artificial sequence
  peptide
RALENATRDXFIICPIIDMA

SEQ ID NO: 53 - protein, 14 aa, Artificial sequence
  peptide
LLSLQEPGSKTXSK

SEQ ID NO: 54 - protein, 16 aa, Artificial sequence
  peptide
EHSTDDXASFSRALEN

SEQ ID NO: 55 - protein, 14 aa, Artificial sequence
  peptide
LKKRGILSPAQLLS

SEQ ID NO: 56 - protein, 16 aa, Artificial sequence
  peptide
SGVIARAAEIMETSIQ

SEQ ID NO: 57 - protein, 14 aa, Artificial sequence
  peptide
PPVREVTRHVIQVS

SEQ ID NO: 58 - protein, 15 aa, Artificial sequence
  peptide
PRQQMNGLTSFLDAS

SEQ ID NO: 59 - protein, 15 aa, Artificial sequence
  peptide
LTALHTLWLREHNRL

SEQ ID NO: 60 - protein, 15 aa, Artificial sequence
  peptide
HNRLAAALKALNAHW

SEQ ID NO: 61 - protein, 14 aa, Artificial sequence
  peptide
ARKVVGALHQIITL

SEQ ID NO: 62 - protein, 16 aa, Artificial sequence
  peptide
LPGLWLHQAFFSPWTL

SEQ ID NO: 63 - protein, 17 aa, Artificial sequence
  peptide
MNEELTERLFVLSNSST

SEQ ID NO: 64 - protein, 11 aa, Artificial sequence
  peptide
LDLASINLQRG

SEQ ID NO: 65 - protein, 16 aa, Artificial sequence
  peptide
RSVADKILDLYKHPDN

SEQ ID NO: 66 - protein, 14 aa, Artificial sequence
  peptide
IDVWLGGLAENFLP

SEQ ID NO: 67 - protein, 12 aa, Artificial sequence
  peptide
QQQHERRKQERK

SEQ ID NO: 68 - protein, 14 aa, Artificial sequence
  peptide
PTKEIEIQVDWNSE

SEQ ID NO: 69 - protein, 780 aa, Homo sapiens
MAAPGGRSEPPQLPEYSCSYMVSRPVYSELAFQQQHERRLQERKTLRESLAKCCSCSRKRAFGVLKTLVPILEWLPKYRVKEWLLSDVISGVSTGLVATLQGMAYALLAAVPVGYGLYSAFFPILTYFIFGTSRHISVGPFPVVSLMVGSVVLSMAPDEHFLVSSSNGTVLNTTMIDTAARDTARVLIASALTLLVGIIQLIFGGLQIGFIVRYLADPLVGGFTTAAAFQVLVSQLKIVLNVSTKNYNGVLSIIYTLVEIFQNIGDTNLADFTAGLLTIVVCMAVKELNDRFRHKIPVPIPIEVIVTIIATAISYGANLEKNYNAGIVKSIPRGFLPPELPPVSLFSEMLAASFSIAVVAYAIAVSVGKVYATKYDYTIDGNQEFIAFGISNIFSGFFSCFVATTALSRTAVQESTGGKTQVAGIISAAIVMIAILALGKLLEPLQKSVLAAVVIANLKGMFMQLCDIPRLWRQNKIDAVIWVFTCIVSIILGLDLGLLAGLIFGLLTVVLRVQFPSWNGLGSIPSTDIYKSTKNYKNIEEPQGVKILRFSSPIFYGNVDGFKKCIKSTVGFDAIRVYNKRLKALRKIQKLIKSGQLRATKNGIISDAVSTNNAFEPDEDIEDLEELDIPTKEIEIQVDWNSELPVKVNVPKVPIHSLVLDCGAISFLDVVGVRSLRVIVKEFQRIDVNVYFASLQDYVIEKLEQCGFFDDNIRKDTFFLTVHDAILYLQNQVKSQEGQGSILETITLIQDCKDTLELIETELTEEELDVQDEAMRTLAS

(19) United States
(12) Patent Application Publication
(10) Pub. No.: US 2016/0022788 A1
(43) Pub. Date: Jan. 28, 2016

(54) COMPOSITIONS AND METHODS FOR TREATING AUTOIMMUNE DISEASES

(71) Applicant: Dorina AVRAM, Albany, NY (US)

(72) Inventor: Dorina Avram, Gainesville, FL (US)

(73) Assignee: Albany Medical College, Albany, NY (US)

(21) Appl. No.: 14/775,393

(22) PCT Filed: Mar. 12, 2014
(86) PCT No.: PCT/US2014/024615

§ 371 Date: Sep. 11, 2015

Related U.S. Application Data
(60) Provisional application No. 61/777,590, filed on Mar. 12, 2013.

(51) Int. Cl. A61K 39/00

(52) U.S. Cl. CPC A61K 39/0008; A61K 2039/55505

(57) ABSTRACT

The present invention provides compositions comprising immunologically effective amounts of one or more autoimmune disease associated antigens or antigenic fragments or derivatives thereof and one or more Th2 promoting adjuvants. The compositions may optionally comprise one or more Th2 promoting TLR2 ligands. The invention further provides methods of treating or preventing an autoimmune disease, such as multiple sclerosis, comprising administering to a subject in need thereof an immunologically-effective amount of an autoimmune disease associated antigen, a Th2 promoting adjuvant and optionally one or more Th2 promoting TLR2 ligands which overall causes re-routing of the harming immune cells to places where they can be of no harm.

### CROSS REFERENCE TO RELATED APPLICATIONS

This application claims the benefit of U.S. Appl. No. 61/777,590, filed Mar. 12, 2013. The content of the aforesaid application is relied upon and incorporated by reference in its entirety.

### STATEMENT OF FEDERALLY SPONSORED RESEARCH AND DEVELOPMENT

This invention was made with government support under Grant No. AI067846 awarded by the National Institutes of Health. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One (77,010 Byte ASCII (Text)) file named “Sequence_listing_ST25.txt,” created on Mar. 11, 2014.

### FIELD OF THE INVENTION

The field of the invention generally relates to the fields of immunology, autoimmunity and medicine. In particular, the field of the invention relates to compositions and methods to treat autoimmune diseases, particularly multiple sclerosis.

### BACKGROUND

Following activation, naïve CD4⁺ T cells differentiate into distinct T-helper subsets, each producing their own cytokines and transcription factors and performing specific biological functions. Th1 subset produces interferon gamma (IFNγ) and controls the immune response to intracellular pathogens, and is governed by the transcription factor T-bet (Szabo, S. J., et al., Cell 100, 655-669 (2000)), while Th2 lineage is controlled by GATA3 (Zheng, W. & Flavell, R. A., Cell 89, 587-596 (1997)) and produces IL-4, IL-5 and IL-13 during anti-parasite and allergic responses (reviewed in O'Shea, J. J. & Paul, W. E., Science 327, 1098-1102 (2010)). Th17 lineage is critical for the immune responses to extracellular bacteria and fungi, produces IL-17 and is controlled by Roryt (Ivanov, I I, et al., Cell 126, 1121-1133 (2006)). Th17 cells are also critical in the autoimmune response in multiple sclerosis (MS) and its mouse model of EAE, with the major player being GM-CSF (Codarri, L., et al., Nat Immunol 12, 560-567 (2011); El-Behi, M., et al. Nat Immunol 12, 568-575 (2011)). The recently identified subset, T follicular helper (Tfh), produces IL-21, is controlled by Bcl6 and plays a critical role in supporting class switch recombination in B cells (reviewed in Crotty, S., Annu Rev Immunol 29, 621-663 (2011)).

Th1, Th2 and Th17 subsets were once deemed highly stable and the alternate lineage cytokines were thought to be inhibitory, such as IFNγ and IL-4 blocking Th17 differentiation and IL-17 production (Harrington, L. E., et al., Nat Immunol 6, 1123-1132 (2005)). However the functional plasticity of the T-helper lineages has become increasingly evident, particularly during in vivo immune responses (reviewed in O'Shea, J. J. & Paul, W. E., Science 327, 1098-1102 (2010); Zhou, L. et al., Immunity 30, 646-655 (2009); Nakayamada, S., et al. Curr Opin Immunol 24, 297-302 (2012)). T-helper cells were found able to express alternative lineage cytokines, such as Th17 cells expressing IFNγ (Lee, Y. K., et al., Immunity 30, 92-107 (2009); Hirota, K., et al., Nat Immunol 12, 255-263 (2011)), or Th2 cells acquiring a Th1 phenotype (Hegazy, A. N., et al. Immunity 32, 116-128 (2010)), or converting into Tfh cells (Zaretsky, A. G., et al., J Exp Med 206, 991-999 (2009)). Tfh cells have the highest plasticity of all sets, being able to convert to Th1, Th2 and Th17 (Lu, K. T., et al., Immunity 35, 622-632 (2011)). In terms of plasticity, the in vivo effects of IL-4 on Th17 immune responses remain largely undefined.

Most human T cell-mediated autoimmune diseases occur spontaneously and are characterized by an insidious onset. Multiple sclerosis is a chronic, and often debilitating disease affecting the central nervous system (brain and spinal cord). Multiple sclerosis affects more than 1 million people worldwide and is the most common neurological disease among young adults, particularly women. Multiple sclerosis attacks the nervous system resulting in myelin sheaths surrounding neuronal axons to be destroyed. This demyelinization can cause weakness, impaired vision, loss of balance, and poor muscle coordination. Multiple sclerosis can have different patterns, sometimes leaving patients relatively well after episodes of acute worsening, sometimes leading to progressive disability that persists after episodes of worsening. Current therapies for multiple sclerosis are unsatisfactory.

The evidence supporting the view that multiple sclerosis is due to immunologic mechanisms comes from histological analysis of multiple sclerosis in humans, as well as work on experimental allergic encephalomyelitis (EAE) in animals. With respect to histological analysis, lesions found in the white matter of patients with multiple sclerosis frequently reveal lymphocyte infiltrates. This underscores the inflammatory cellular immune model for the disease.

Animal studies with EAE also provide support for the model. EAE demonstrates significant similarities to multiple sclerosis. See generally, E. Alvord, Experimental Allergic Encephalomyelitis: A Useful Model for Multiple Sclerosis, Progress in Clinical and Biological Research, E. C. Alvord et al. (Eds.) (New York, N.Y.) (1984). EAE is an autoimmune disease mediated by antigen-specific, class II-restricted CD4⁺ T cells. See S. Zamvil and L. Steinman, Ann. Rev. Immunol. 8:579-621(1990). Like multiple sclerosis, EAE is an acute, inflammatory, demyelinating disease with certain forms characterized by relapsing paralysis.

It is shown herein that immunization under Th2 conditions, viz., by administration of a vaccine composition comprising myelin oligodendrocyte glycoprotein and alum, plus a TLR2 ligand, following EAE induction, causes a dramatic amelioration of EAE in wild type mice. It is demonstrated that Th17 cells produced IL-4, without any impact on IL-17 and GM-CSF. After treatment, the immune cells maintained their ability to produce Th17 cytokines, thereby maintaining their ability to fight infections. Unexpectedly, the major physiological consequence of IL-4 production during EAE was the diverted migration of the T-helper cells from the draining lymph nodes (dLNS)-CNS route to the mesenteric lymph nodes (mLNs)-gut. It is believed that the diverted migration of the cells is caused by upregulation of the gut homing receptors CCR9 and integrin a4b7. It is also shown that dendritic cells of the EAE wild type mice treated in Th2 conditions present elevated levels of Radh activity, implicated in retinoic acid production, known to imprint gut homing on T cells. Importantly the treatment, though it resulted in gut migration of T cells, it did not cause overt colitis. Additionally, it is shown that administration of high levels of Vitamin A also ameliorate the disease scores and onset, and without being bound by theory, by reducing the effector CD4⁺ T cells in the CNS and draining lymph nodes and increasing them in mesenteric lymph nodes.

### SUMMARY

In some embodiments, the present invention provides methods and compositions to treat autoimmune diseases, such as, for example, multiple sclerosis.

In one aspect, the invention provides compositions comprising an immunologically effective amount of one or more autoimmune disease associated antigens or antigenic fragments or derivatives thereof and one or more Th2 promoting adjuvants. In some embodiments, the compositions further comprise one or more additional Th2 promoting TLR2 ligands. In some embodiments, the TLR2 ligand is selected from the group consisting of Pam3CysSerLys4 (Pam3CSK4), 3-palmytoil-s-glycerylcysteine, Pam2CSK4, diacetylated lipopetide FSL-1 (Pam2CGDPKHPKSF), lipoteichoic acid, peptidoglycan and combinations thereof. In some embodiments, the compositions are pharmaceutical compositions further comprising a pharmaceutically acceptable carrier. In some embodiments, the compositions are formulated and administered as vaccine compositions.

In another aspect, the invention provides methods of treating or preventing an autoimmune disease, comprising administering to a subject in need thereof an immunologically effective amount of an autoimmune disease associated antigen, and one or more Th2 promoting adjuvants and optionally one or more Th2 promoting TLR2 ligands. In some embodiments, the autoimmune disease associated antigen, the Th2 promoting adjuvant and the Th2 promoting TLR2 ligand are administered together. In some embodiments, they are administered separately. In some embodiments, the autoimmune disease associated antigen and Th2 promoting adjuvant are administered together while the Th2 promoting TLR2 ligand is administered separately.

In some embodiments, the autoimmune disease is multiple sclerosis.

In some embodiments, the one or more autoimmune disease associated antigens or antigenic fragments or derivatives thereof is selected from the group consisting of myelin basic protein, myelin associated glycoprotein, alphaB-crystallin, S100beta, proteolipid protein (PLP) and myelin oligodendrocyte glycoprotein (MOG).

In some embodiments, the one or more autoimmune disease associated antigens is selected from the group consisting of MOG₃₅₋₅₅ mouse fragment, MEVGWYRSPFSRVVHLYRNGK (SEQ ID NO:13); MOG human fragment, MEVGWYRPPFSRVVHLYRNGK (SEQ ID NO:14); MAG₂₈₇₋₂₉₅ human fragment, SLLLELEEV (SEQ ID NO:15); MAG₂₈₇₋₂₉₅ mouse fragment, SLYLDLEEV (SEQ ID NO:16); MAG₅₀₉₋₅₁₇ mouse and human fragment, LMWAKIGPV (SEQ ID NO:17); MAG₅₅₆₋₅₆₄, human fragment, VLFSSDFRI (SEQ ID NO:18); MAG₅₅₆₋₅₆₄, mouse fragment, VLYSPEFRI (SEQ ID NO:19); MBP human fragment, SLSRFSWGA (SEQ ID NO:20); MBP mouse fragment, SLSRFSWGG (SEQ ID NO:21); MOG mouse and human fragment, KVEDPFYWV (SEQ ID NO:22); MOG mouse and human fragment, RTFDPHFLRV (SEQ ID NO:23); MOG mouse and human fragment, FLRVPCWKI (SEQ ID NO:24); MOG mouse and human fragment, KITLFVIVPV (SEQ ID NO:25); MOG mouse and human fragment, VLGPLVALI (SEQ ID NO:26); MOG mouse and human fragment, TLFVIVPVL (SEQ ID NO:27); MOG mouse and human fragment, RLAGQFLEEL (SEQ ID NO:28); PLP₈₀₋₈₈ mouse and human fragment, FLYGALLLA (SEQ ID NO:29); and combinations thereof.

In some embodiments, the one or more autoimmune disease associated antigens or antigenic fragments or derivatives thereof is present in the composition at about a 1:1 ratio by weight with the adjuvant. In some embodiments, the adjuvant increases production of IL-4 upon administration in a subject and causes re-routing of the harming immune cells to places where they can be of no harm. In some embodiments, the adjuvant is an aluminum containing adjuvant.

In some embodiments, the adjuvant is selected from the group consisting of AlNa(SO₄)₂, AlNH₄(SO₄), aluminum hydroxide, aluminum phosphate, potassium aluminum sulfate (“alum”), and combinations thereof.

In some embodiments, the Th2 promoting TLR2 ligand is selected from the group consisting of Pam3CysSerLys4 (Pam3CSK4), 3-palmytoil-s-glycerylcysteine, Pam2CSK4, diacetylated lipopetide FSL-1 (Pam2CGDPKHPKSF), lipoteichoic acid, peptidoglycan and combinations thereof.

In some embodiments, the subject is further administered from about 3000-25000 mcg retinol activity equivalents (RAE) per day. In some embodiments, the subject is administered about 7000 mcg of retinol activity equivalents (RAE) per day. In some embodiments, the retinol activity equivalents (RAE) are administered in an oral dosage form in a pharmaceutical composition.

In another aspect, the invention provides a method of treating or preventing an autoimmune disease comprising administering to a subject in need thereof from about 3000-25000 mcg of retinol activity equivalents (RAE) per day. In some embodiments, the subject is administered about 7000 mcg of retinol activity equivalents (RAE) per day. In some embodiments, the autoimmune disease is multiple sclerosis.

In another aspect, the invention provides a pharmaceutical composition for treating autoimmune disease in a subject, comprising from about 3000-25000 mcg of retinol activity equivalents (RAE) in combination with a pharmaceutically acceptable carrier.

In some embodiments, the composition comprises about 7000 mcg of retinol activity equivalents (RAE). In some embodiments, the autoimmune disease is multiple sclerosis.

It is to be understood that both the foregoing general description and the following detailed description are exemplary, and thus do not restrict the scope of the invention.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1. Treatment of EAE wild type mice in Th2 conditions reduced the clinical scores. EAE disease scores in wild type (WT) mice immunized with MOG₃₅₋₅₅ in CFA followed by treatment with either MOG₃₅₋₅₅ in alum, plus Pam3CSK4 (red) (WT MOG/Alum) or alum alone (black) (WT/Alum). Treatment was initiated on day 7 post EAE induction. Significance was determined by two-tailed student's t test; p-value ≦0.05 after day 12; n=7 (mean±SEM). This is a representative experiment out of three.

FIG. 2. Treatment of EAE wild type mice with MOG/Alum induces production of IL-4 without inhibition of IL-17 and GM-CSF production. EAE wild type mice were treated with either MOG₃₅₋₅₅ in alum, plus Pam3CSK4 or alum alone on day 7 post-EAE induction. (A-C) Flow cytometry analysis of IL-4 (A), IL-17 (B) and GM-CSF (C) in gated CD4⁺ T cells from dLNs (left) and mLNs (right). Data is representative of three pairs of mice.

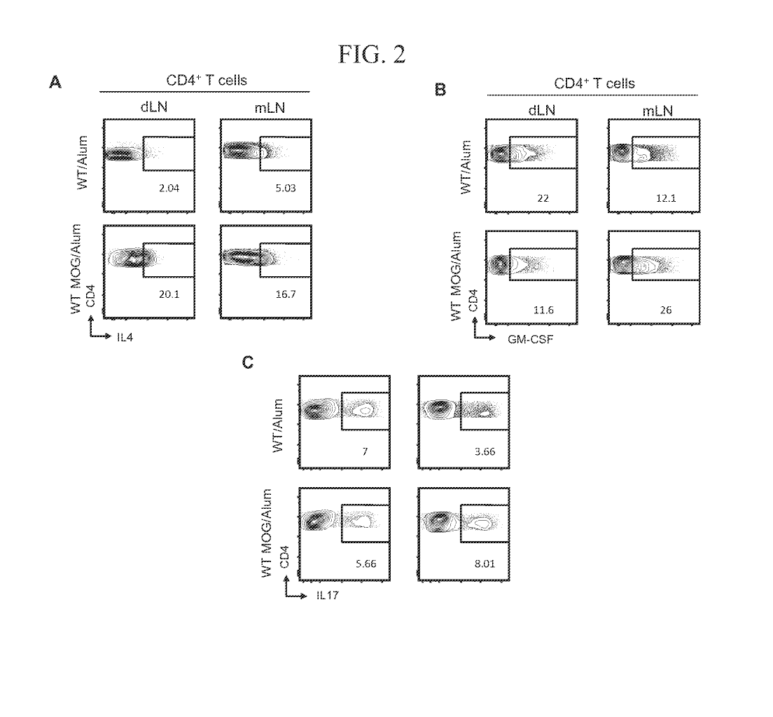

FIG. 3. Treatment of EAE wild type mice in Th2 conditions reduces infiltration of CD4⁺ T cells in the CNS and causes their redistribution from dLNs-CNS route to the mLNs-gut. (A) Absolute numbers of CD4⁺ T cells from dLNs and mLNs from EAE WT/Alum (black), and EAE WT MOG/Alum mice (red) on day 12 after EAE induction. Two-tailed student's t test was applied to determine significance (* indicates p-value ≦0.01; n=6) (mean±SEM). (B) Gross anatomy of dLNs, mLNs and Peyer's patches on day 12 following EAE induction in MOG/Alum- or Alum-treated EAE wild type mice. (C) FACS analysis of frequencies of CD4⁺ T cells in the CNS (left), Peyer's patches (center) and small intestine lamina propria (SILP) (right) in EAE wild type mice treated as indicated, on day 18 following EAE induction. Data is representative for four pairs of mice.

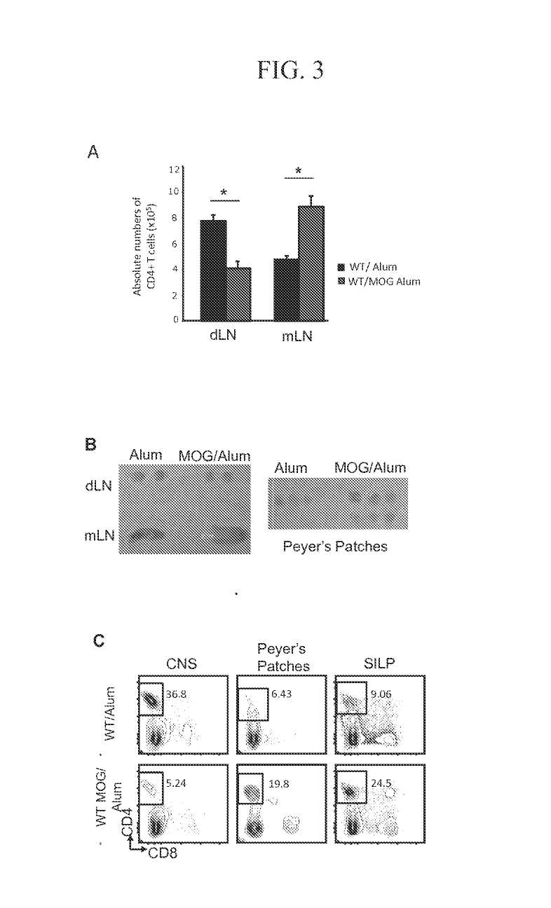

FIG. 4. EAE wild type mice treated in Th2 conditions upregulate gut-homing receptors and express normal levels of CCR6. (A) Flow cytometry analysis of CCR6 (A), CCR9 (B) and integrin α4β7 levels on CD4⁺ T cells from dLNs and mLNs of EAE mice treated with MOG/Alum (red) or Alum (black). Data is representative for four pairs of mice.

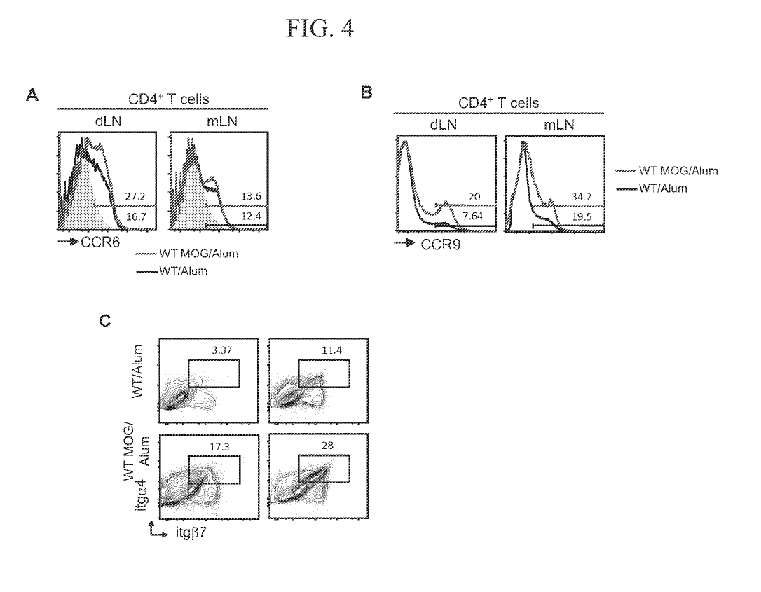

FIG. 5. Dendritic cells of EAE wild type mice treated in Th2 conditions express elevated levels of Raldh activity. ALDH activity in dendritic cells (CD11c⁺ CD103⁺ CD3⁻) from dLNs and mLNs of EAE mice treated with MOG/Alum (red) or Alum (black). The gray shaded area represents DEAB treated samples. Data is representative for three pairs of mice.

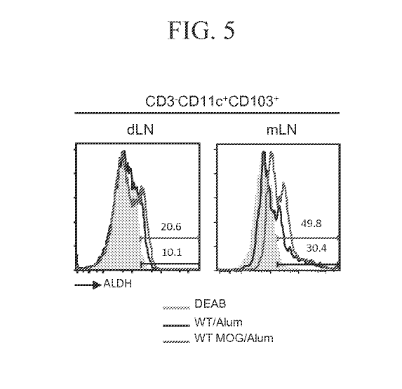

FIG. 6. EAE wild type mice treated in Th2 conditions do not show signs of inflammation in the small intestine. H&E staining of small intestine sections of MOG/Alum- and Alum-treated EAE wild type mice on day 30 following EAE induction. Data is representative for three pairs of mice.

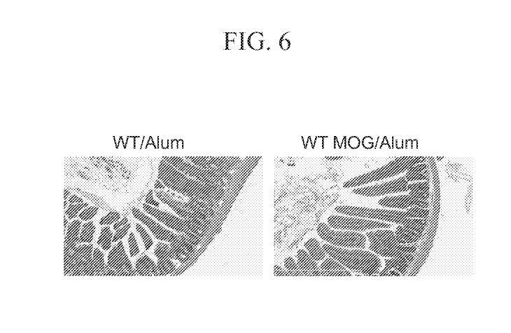

FIG. 7. Mice on high Vitamin A diet have reduced EAE severity and delayed onset. Mice were placed on high Vitamin A diet (200 U/g) versus normal diet, which has 20 U/g, at 3-4 weeks of age, when weaned. Disease was induced at 10 weeks of age and mice were further maintained on the High Vitamin A versus regular diet. Data is representative for 6 pairs of mice.

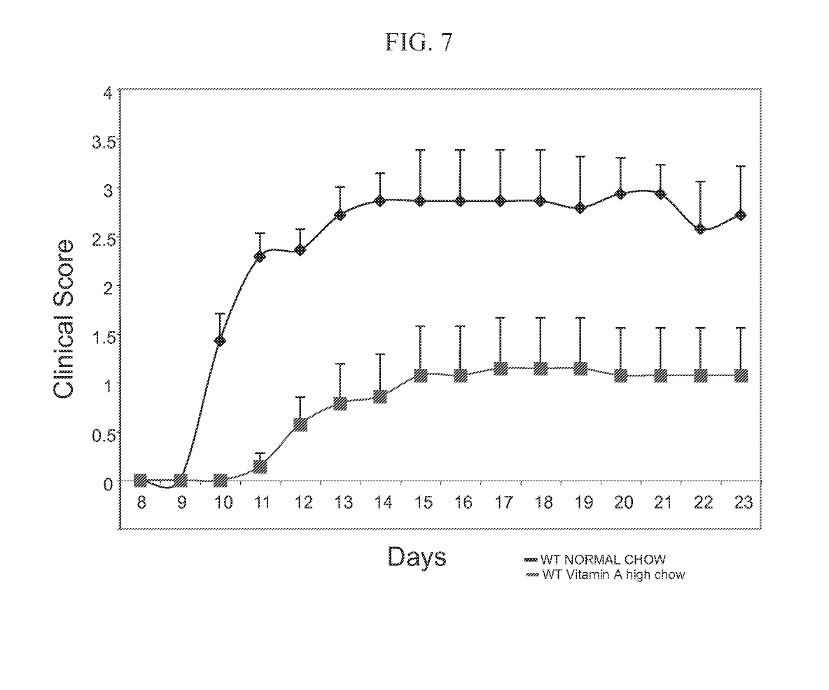

FIG. 8. Mice on High Vitamin A diet have reduced infiltration of CD4⁺ T cells in the CNS and redistribution of the CD4⁺ T cells from dLNs-CNS route to the mLNs-gut, associated with upregulation of gut homing markers. (A) FACS analysis of frequencies of CD4⁺ T cells in the CNS (left), Peyer's patches (center) and small intestine lamina propria (SILP) (right) in EAE wild type mice treated as in FIG. 7, on day 19 following EAE induction. (B) Flow cytometry analysis of surface CCR9, CCR6 (left), and integrin α4β7 (right) on CD4⁺ T cells from dLNs and mLNs of EAE wild type as in FIG. 7, on day 19 following EAE induction. Data is representative for three pairs of mice.

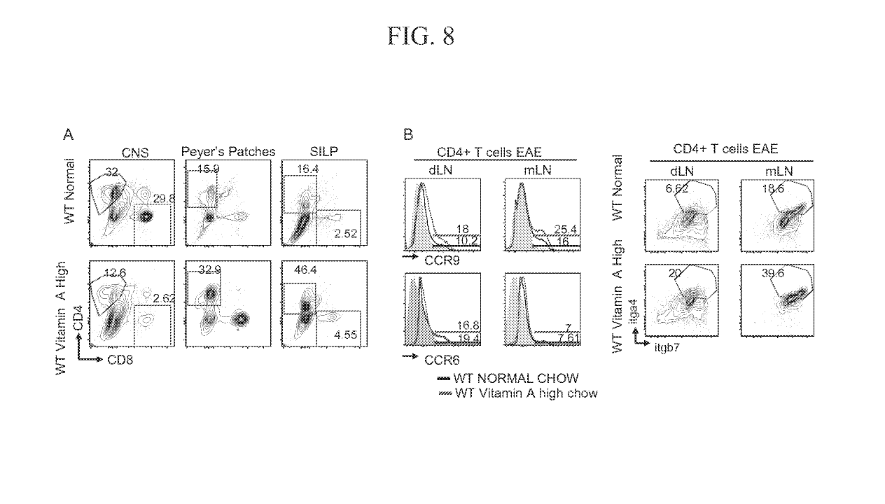

FIG. 9. Mice on High Vitamin A diet have reduced percentages of IL-17 and Gm-CSF-producing CD4⁺ T cells in the CNS and draining lymph nodes (dLNs), however larger percentages in mesenteric lymph nodes (A-C) Flow cytometry analysis of IL-17 and GM-CSF in gated CD4⁺ T cells from CNS (A), dLNs (B), and mLNs (C). Data is representative of three pairs of mice.

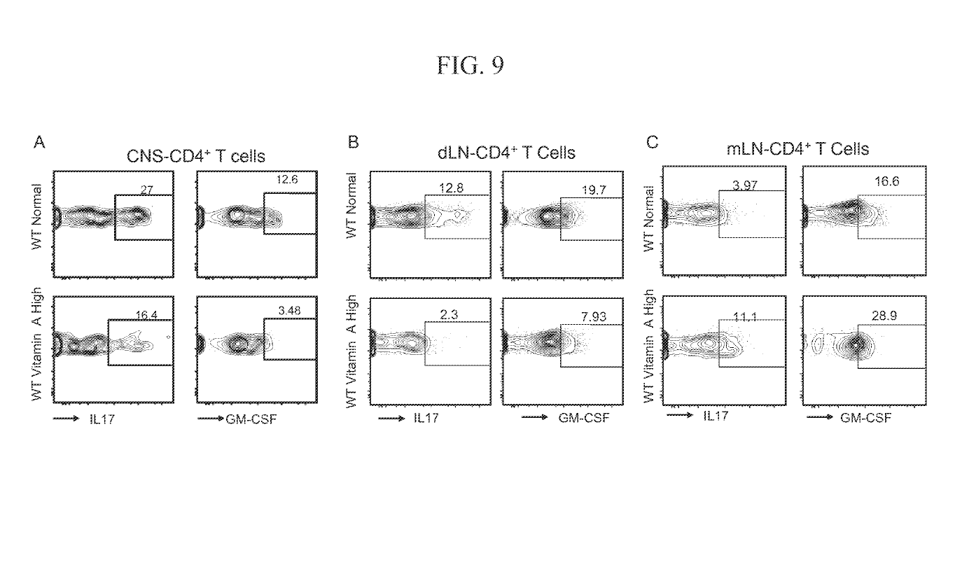

FIG. 10. Treatment of EAE wild type mice with MOG₃₅₋₅₅ in incomplete Freund's adjuvant (MOG/IFA) reduced the clinical scores and decreases the CD4⁺ T cells infiltrating the CNS. (A) EAE disease scores in wild type (WT) mice immunized with MOG₃₅₋₅₅ in CFA followed by treatment with MOG₃₅₋₅₅ in IFA; WT MOG/IFA (red) or WT untreated (black). Treatment was initiated on day 7 post EAE induction. Significance was determined by two-tailed student's t test; p-value ≦0.05 after day 12; n=5 (mean±SEM). (B) FACS analysis of frequencies of CD4⁺ T cells in the CNS in EAE wild type mice treated as indicated, on day 18 following EAE induction. Data is representative for four pairs of mice.

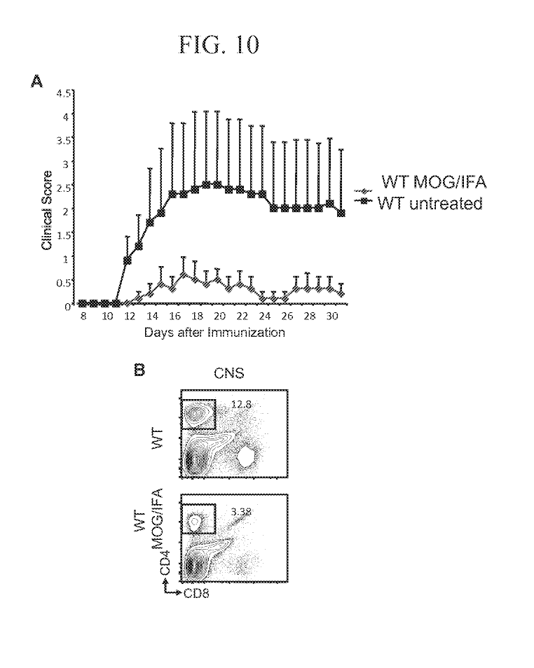

FIG. 11. Treatment of EAE wild type mice with MOG/IFA does not induce production of IL-4, but diminishes production of IL-17 and GM-CSF. (A) Flow cytometry analysis of IL-17, GM-CSF and IL-4 in gated CD4⁺ T cells from dLNs (left) and mLNs (right) of EAE wild type mice treated with MOG₃₅₋₅₅ in IFA, or left untreated. Data is representative of three pairs of mice. (B) CCR6 on gated CD4⁺ T cells from dLNs (left) and mLNs (right) of the indicated mice.

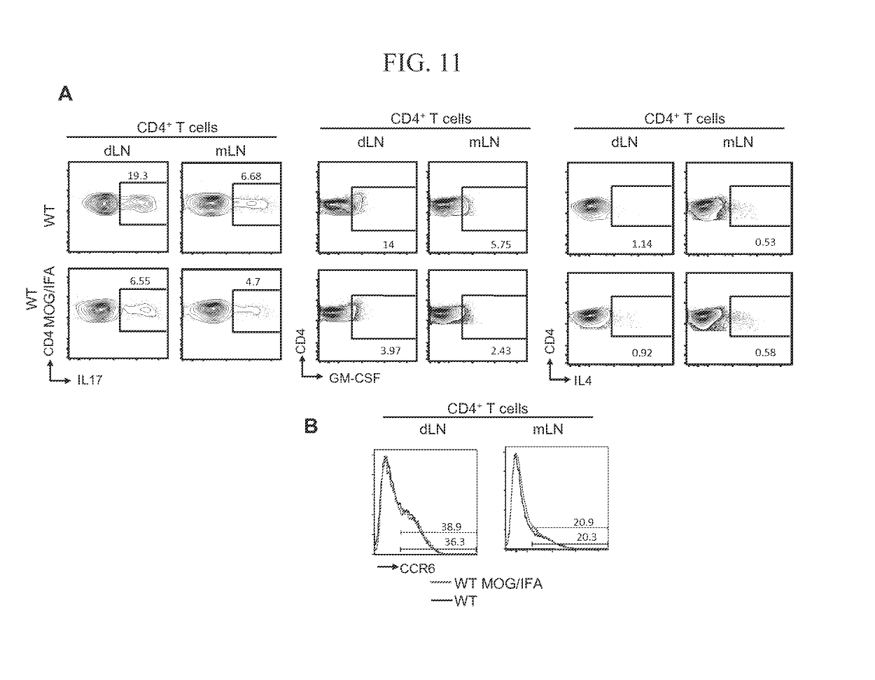

FIG. 12. CD4⁺ T cells of EAE wild type mice treated with MOG/IFA do not express elevated levels of gut-homing receptors and do not accumulate to the Peyer's patches or small intestine lamina propria (SILP). (A) FACS analysis of frequencies of CD4⁺ T cells in the Peyer's patches and small intestine lamina propria (SILP) in EAE wild type mice treated as indicated, on day 18 following EAE induction. Data is representative for three pairs of mice. (B and C) Flow cytometry analysis of CCR9 (B) and integrin α4β7 (C) levels on CD4⁺ T cells from dLNs and mLNs of EAE mice treated with MOG/IFA (red) or untreated (black). Data is representative for three pairs of mice.

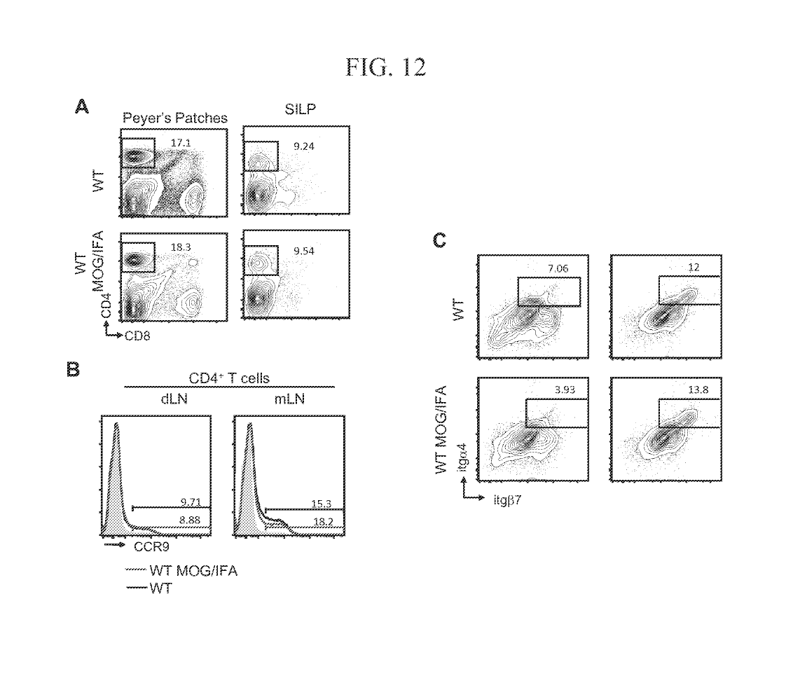

FIG. 13. Model for re-routing of CD4⁺ T cells from the dLNs/CNS to mLNs/gut through increased plasticity of Th17 cells following IL-4 induction during EAE. (A) During EAE induction, following activation in the draining lymph node, wild type CD4⁺ T cells downregulated GATA3, upregulate Roryt, which induces GM-CSF and IL-17 expression, upregulation of CCR6 and migration of CD4⁺ T cells and other immune cells to the CNS. Immunization in Th2 conditions causes production of IL-4, together with IL-17, and more importantly GM-CSF. Production of IL-4 together with GM-CSF results in the upregulation of Raldh2 expression and activity in dendritic cells, which produce elevated levels retinoic acid (RA). RA induces expression of CCR9 and α4β7 on T helper cells, which causes their diverted migration away from dLNs-CNS route toward the mLNs-small intestine. This provides protection from EAE, despite the fact that these cells express normal levels of CCR6.

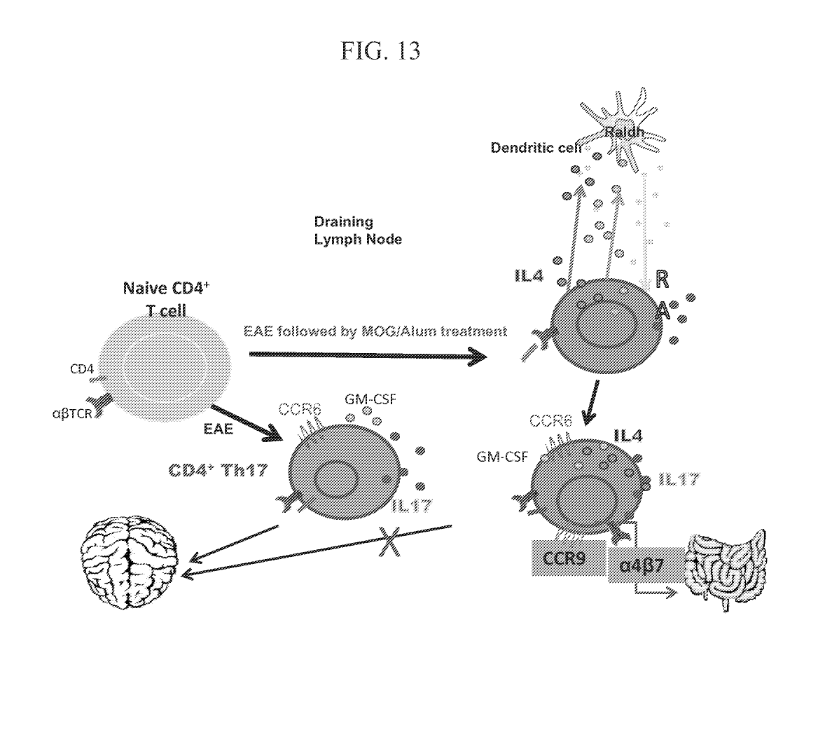

### DETAILED DESCRIPTION

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, the exemplary methods, devices, and materials are now described.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of “or” means “and/or” unless stated otherwise. The use of “a” herein means “one or more” unless stated otherwise or where the use of “one or more” is clearly inappropriate. The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of” In some embodiments herein, “about” refers to ±10% of the numerical value recited.

“Antigen” as used herein refers to all, part, fragment, or segment of a molecule that can induce an immune response in a subject or an expansion of non-pathogenic cells. An “autoimmune disease associated antigen” as used herein is a “self” antigen that is recognized by a subject's own immune system. An autoimmune disease associated antigen has potential to stimulate production of antibodies and lead to autoimmune disease.

An “effective amount” or “immunologically effective amount” is an amount sufficient to achieve the intended purpose, e.g., modulation of T cell activity or T cell populations and/or amelioration of autoimmune disease or symptoms. As described herein in detail, the effective amount, or dosage, depends on the purpose and the antigen and can be determined according to the present disclosure.

The terms “inhibiting,” “treating,” “reducing,” or “preventing,” or any variation of these terms, when used in the claims and/or the specification includes any measurable decrease or complete inhibition to achieve a desired result.

The present invention provides methods and compositions to treat autoimmune diseases.

In one embodiment, the invention provides a composition comprising an immunologically effective amount one or more autoimmune disease associated antigens or antigenic fragments or derivatives thereof and one or more Th2 promoting adjuvants. In some embodiments, the compositions further comprise one or more Th2 promoting TLR2 ligands such as Pam3CysSerLys4 (Pam3CSK4), 3-palmytoil-s-glycerylcysteine, Pam2CSK4, diacetylated lipopetide FSL-1 (Pam2CGDPKHPKSF), lipoteichoic acid, and peptidoglycan and combinations thereof. In some embodiments, the compositions are pharmaceutical compositions comprising a pharmaceutically acceptable carrier. In some embodiments, the compositions are formulated as vaccine compositions to be administered to a subject in need of treatment.

In some embodiments, the invention provides a method of treating or preventing an autoimmune disease comprising administering to a subject in need thereof an immunologically effective amount of one or more autoimmune disease associated antigens, one or more Th2 promoting adjuvants and optionally one or more Th2 promoting TLR2 ligands.

An autoimmune disease may include, but is not limited to multiple sclerosis, experimental autoimmune encephalomyelitis (EAE), diabetes melitus, transplantation rejection, premature ovarian failure, scleroderm, Sjogren's disease, lupus, vilelego, alopecia (baldness), polyglandular failure, Grave's disease, hypothyroidism, polymyosititis, pemphigus, autoimmune hepatitis, hypopituitarism, myocardititis, thyroiditis, Addison's disease, autoimmune skin diseases, uveititis, pernicious anemia, hypoparathyroidism, and/or rheumatoid arthritis.

In some embodiments of the invention, the autoimmune disease to be treated is multiple sclerosis. In some embodiments, the compositions and methods are useful in treating EAE.

Without being bound by theory as to how the invention works, it is believed that the compositions and methods of the invention cause diverted migration of pathogenic CD4⁺ T cells away from the site of the autoimmune disease. Most notably for multiple sclerosis, it is believed that the compositions and methods when administered to a subject cause diverted migration of pathogenic CD4⁺ T cells from the draining lymph nodes (dLNs) and the central nervous system (CNS) to the mesenteric lymph nodes (mLNs) and gut without causing overt colitis, resulting in significant amelioration of disease.

Shown herein in the Examples are results with mice having experimental autoimmune encephalomyelitis (EAE) that experienced significant delay in the progression of their disease and reduced severity of the disease after they were immunized with myelin oligodendrocyte glycoprotein peptide (MOG) combined with Th2 immunological adjuvant(s). Instead of causing increased demyelination of the central nervous system (CNS) as conventional wisdom would expect, the Th2 treatment instead surprisingly and unexpectedly altered T-helper cell trafficking to the gut, where they are harmless.

It is shown herein that Th-17 cells responsible for the auto-immune response in EAE could be made to express cytokines normally associated with other T-helper lineage cells. The Th2 treatment took advantage of this T-helper cell plasticity, causing abnormal production of Th-2 lineage interleukin-4 (IL4) by the Th-17 cells. This triggered an overriding expression of gut homing receptors, protecting the central nervous system from the auto-immune response.

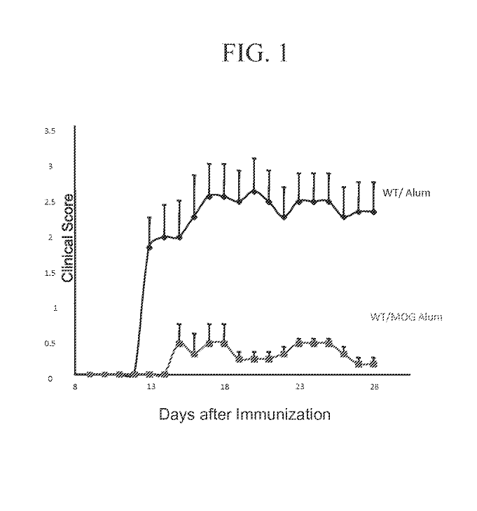

These surprising and unexpected results demonstrate that such treatments can inhibit the progression of autoimmune diseases such as multiple sclerosis. Unlike MS treatments that seek to block infiltration of T cells into the CNS, in some embodiments, a single immunization can offer long-term protection after the onset of the auto-immune disorder by causing an epigenetic change in the destructive T-helper cells. Specifically, following the treatment, the disease scores at the peak of disease were found reduced to 0-0.5 from 3.5 in the untreated group (see the FIG. 1 and the Examples below).

In some embodiments of the invention, the autoimmune disease associated antigen can be a polypeptide, peptide, nucleic acid, carbohydrate, lipid or other molecule that provokes or induces an antigenic response against self, generally referred to as self-antigens. In some embodiments, the autoimmune disease associated antigen corresponds to the native or natural self-antigen. In other embodiments, an antigenic fragment or antigenic derivative can be used.

In some embodiments, the compositions of the invention comprise an autoimmune disease associated antigen or an antigenic fragment or derivative thereof, such as an epitope, or a mimic thereof, involved in the autoimmune response to be treated or prevented. In some embodiments, the autoimmune disease associated antigen is an antigenic fragment, epitope, or peptide of a protein, carbohydrate, or lipid expressed by certain cells of a subject. Various proteins or epitopes have been identified for a variety of autoimmune conditions and are useful in the present invention.

Polypeptides and peptide autoimmune disease associated antigens of the invention can be modified by various amino acid deletions, insertions, and/or substitutions. In particular embodiments, modified polypeptides and/or peptides are capable of modulating an immune response in a subject. As used herein, a “protein” or “polypeptide” or “peptide” refers to a molecule comprising at least five amino acid residues. In some embodiments, a wild-type version of a protein or peptide is employed, however, in many embodiments of the invention, a modified protein or polypeptide is employed.

A “modified protein,” “modified polypeptide,” or “modified peptide” refers to a protein or polypeptide whose chemical structure, particularly its amino acid sequence, is altered with respect to the wild-type protein or polypeptide. In some embodiments, a modified protein or polypeptide or peptide has at least one modified activity or function (recognizing that proteins or polypeptides or peptides may have multiple activities or functions). It is specifically contemplated that a modified protein or polypeptide or peptide may be altered with respect to one activity or function yet retains a wild-type activity or function in other respects, such as immunogenicity or ability to interact with other cells of the immune system in the context of the compositions of the invention when administered to a subject.

In some embodiments, the autoimmune disease associated antigen is a multiple sclerosis associated antigen. In some embodiments, the multiple sclerosis associated antigen is selected from the group consisting of myelin basic protein (MBP), myelin associated glycoprotein (MAG), alphaB-crystallin, S100beta, proteolipid protein (PLP) and myelin oligodendrocyte glycoprotein (MOG) and combinations thereof. Antigenic fragments and antigenic derivatives of these antigens are also contemplated.

In some embodiments, myelin basic protein (MBP), myelin associated glycoprotein (MAG), alphaB-crystallin, S100beta, proteolipid protein (PLP) and myelin oligodendrocyte glycoprotein (MOG) have the following sequences:

Myelin oligodendrocyte glycoprotein (human; GenBank CAA52617.1): SEQ ID NO:1;
Myelin oligodendrocyte glycoprotein (mouse; GenBank: AAH80860.1): SEQ ID NO:2;
Myelin basic protein (human; Accession No: P02686): SEQ ID NO:3;
Myelin basic protein (mouse; GenBank: AAB59711.1): SEQ ID NO:4;
Myelin associated glycoprotein (human; GenBank: AAH53347.1): SEQ ID NO:5;
Myelin associated glycoprotein (mouse; Accession No.: P20917): SEQ ID NO:6;
S100beta (human; Accession No.: NP_{—}006263): SEQ ID NO:7;
S100beta (mouse; Accession No.: NP_{—}033141): SEQ ID NO:8;
Proteolipid protein (human; GenBank: AAA60117.1): SEQ ID NO:9;
Proteolipid protein (mouse; GenBank: CAA30184.1): SEQ ID NO:10;
AlphaB crystallin (human; Accession No.: 2KLR_A): SEQ ID NO:11; and
AlphaB crystallin (mouse; GenBank: AAH94033.1): SEQ ID NO:12.

In some embodiments, the compositions and methods comprise an antigenic fragment of a multiple sclerosis associated protein. Peptides useful in the compositions and methods of the invention can include any autoreactive peptide. In some embodiments, multiple sclerosis associated antigenic peptides can include, but are not limited to:

MOG₃₅₋₅₅ mouse fragment,
(SEQ ID NO: 13)
MEVGWYRSPFSRVVHLYRNGK;
MOG human fragment
(SEQ ID NO: 14)
MEVGWYRPPFSRVVHLYRNGK;
MAG₂₈₇₋₂₉₅, human fragment,
(SEQ ID NO: 15)
SLLLELEEV;
MAG₂₈₇₋₂₉₅, mouse fragment,
(SEQ ID NO: 16)
SLYLDLEEV;
MAG₅₀₉₋₅₁₇, mouse and human fragment,
(SEQ ID NO: 17)
LMWAKIGPV;
MAG₅₅₆₋₅₆₄, human fragment,
(SEQ ID NO: 18)
VLFSSDFRI;
MAG₅₅₆₋₅₆₄, mouse fragment,
(SEQ ID NO: 19)
VLYSPEFRI;
MBP human fragment,
(SEQ ID NO: 20)
SLSRFSWGA;
MBP mouse fragment,
(SEQ ID NO: 21)
SLSRFSWGG;
MOG mouse and human fragment,
(SEQ ID NO: 22)
KVEDPFYWV;
MOG mouse and human fragment,
(SEQ ID NO: 23)
RTFDPHFLRV;
MOG mouse and human fragment,
(SEQ ID NO: 24)
FLRVPCWKI;
MOG mouse and human fragment,
(SEQ ID NO: 25)
KITLFVIVPV;
MOG mouse and human fragment,
(SEQ ID NO: 26)
VLGPLVALI;
MOG mouse and human fragment,
(SEQ ID NO: 27)
TLFVIVPVL;
MOG mouse and human fragment,
(SEQ ID NO: 28)
RLAGQFLEEL;
PLP₈₀₋₈₈, mouse and human fragment,
(SEQ ID NO: 29)
FLYGALLLA;
MOG fragment
(SEQ ID NO: 30)
HPIRALVGDEVELP;
MOG fragment
(SEQ ID NO: 31)
VGWYRPPFSRVVHLYRNGKD;
MOG fragment
(SEQ ID NO: 32)
LKVEDPFYWVSPGVLVLLAVLPVLLL;

and combinations thereof

In some embodiments, 1, 2, 3, 4, 5, 6, 7, 8, 9, 10, 11, 12, 13, 14, 15, 16, 17, 18, 19, 20 or more peptides can be used in combination. In some embodiments any of SEQ ID NOS: 14, 22, 23, 24-28, 30, 31 and 32 are used in combination with each other or with other multiple sclerosis associated antigenic peptides.

In some embodiments, the autoimmune disease associated antigen is a diabetes melitus associated antigen. In some embodiments, the antigen is selected from the group consisting of insulin (GenBank: AAA59172.1; SEQ ID NO: 33), chromogranin A (GenBank: AAB53685.1; SEQ ID NO: 34), glutamic acid decarboxylase (GenBank: CAB62572.1; SEQ ID NO: 35) and islet-specific glucose-6-phosphatase catalytic subunit-related protein (GenBank: AAF82810.1: SEQ ID NO: 36 and combinations thereof. Antigenic fragments and antigenic derivatives of these antigens are also contemplated. In some embodiments, the antigen can be proinsulin. In some embodiments, the proinsulin antigen can have the sequence MALWMRLLPLLALLALWGPDPAAAFVNQHLCGSHLVEALYLVC GERGFF YTPKTRREAEDLQVGQVELGGGPGAGSLQPLALEGSLQKRGIVEQCCTSIC S LYQLENYCN (SEQ ID NO:37), which can be encoded by a sequence contained in GenBank Accession No. NM000207, the contents of which are incorporated by reference herein. In some embodiments, the insulin antigen comprises the sequence MRLLPLLALLA (SEQ ID NO:38), SHLVEALYLVCGERG (SEQ ID NO:39), or LYLVCGERG (SEQ ID NO:40). In some embodiments, the insulin antigen can have the amino acid sequence GIVEQCCTSICSLYQ (SEQ ID NO:41). Combinations of the above listed antigens are also contemplated.

In some embodiments, the autoimmune disease associated antigen is a rheumatoid arthritis associated antigen. In some embodiments, the rheumatoid arthritis associated antigen can be the peptide (Q/R)(K/R)RAA (SEQ ID NO:42). In some embodiments, the arthritis associated antigen can be type II collagen or a fragment thereof. In some embodiments, the type II collagen fragment is selected from the group consisting of AGERGPPG (SEQ ID NO: 43), AGGFDEKAGGAQLGV (SEQ ID NO:44), VGPAGGPGFPG (SEQ ID NO:45), and a combination thereof.

In some embodiments, the autoimmune disease associated antigen is a myocardititis associated antigen. In some embodiments, the myocardititis associated antigen is myosin or an antigenic fragment or antigenic derivative. In some embodiments, the antigen can be a peptide contained in human myosin (GeneBank Accession No. CAA86293.1; SEQ ID NO:46). In some embodiments, the antigen can be a peptide contained within α-myosin, and can have the sequence Ac-SLKLMATLFSTYASADTGDSGKGKGGKKKG (SEQ ID NO:47; where Ac is an acetyl group), GQFIDSGKAGAEKL (SEQ ID NO:48), DECSELKKDIDDLE (SEQ ID NO:49), and combinations thereof.

In some embodiments, the autoimmune disease associated antigen is a thyroiditis associated antigen. In some embodiments, the antigen is selected from thyroid peroxidase (TPO), thyroglobulin, or Pendrin. In some embodiments, the thyroglobulin antigen can have the sequence, NIFEXQVDAQPL (SEQ ID NO:50), YSLEHSTDDXASFSRALENATR (SEQ ID NO:51), RALENATRDXFIICPIIDMA (SEQ ID NO:52), LLSLQEPGSKTXSK (SEQ ID NO:53), EHSTDDXASFSRALEN (SEQ ID NO:54) and combinations thereof, wherein X is 3,5,3′,5′-tetraiodothyronine (thyroxine). In some embodiments, the TPO antigen can have the sequence LKKRGILSPAQLLS (SEQ ID NO:55), SGVIARAAEIMETSIQ (SEQ ID NO:56), PPVREVTRHVIQVS (SEQ ID NO:57), PRQQMNGLTSFLDAS (SEQ ID NO:58), LTALHTLWLREHNRL (SEQ ID NO:59), HNRLAAALKALNAHW (SEQ ID NO;60), ARKVVGALHQIITL (SEQ ID NO:61), LPGLWLHQAFFSPWTL (SEQ ID NO:62), MNEELTERLFVLSNSST (SEQ ID NO:63), LDLASINLQRG (SEQ ID NO:64), RSVADKILDLYKHPDN (SEQ ID NO:65), IDVWLGGLAENFLP (SEQ ID NO:66) and combinations thereof. The Pendrin antigen can have the sequence QQQHERRKQERK [amino acids 34-44 in human pendrin (GenBank AF030880)] (SEQ ID NO:67), PTKEIEIQVDWNSE [amino acids 630-643 in human pendrin] (SEQ ID NO:68), or NCBI GenBank Accession No. NP_{—}000432.1 (SEQ ID NO:69).

The size of a protein or polypeptide fragment can be of any size, and in some embodiments comprises (wild-type or modified), at least 5 amino acids. In some embodiments, the fragment is at least about 6, 7, 8, 9, 10, 11, 12, 13, 14, 15, 16, 17, 18, 19, 20, 21, 22, 23, 24, 25, 26, 27, 28, 29, 30, 35, 40, 50, 60, 70, 80, 90, 100, 110, 120, 130, 140, 150, 160, 170, 180, 190, 200, 210, 220, 230, 240, 250, 275, 300, 325, 350, 375, 400, 425, 450, 475, or about 500 amino acids or greater, including any range or value derivable therein. In some embodiments, 5, 6, 7, 8, 9, 10 or more contiguous amino acids, including derivatives thereof, and fragments of the autoimmune disease associated antigen, such as those amino acid sequences disclosed and referenced herein, can be used. It is contemplated that polypeptides may be mutated by truncation, rendering them shorter than their corresponding wild-type form, but also they might be altered by fusing or conjugating a heterologous protein sequence with a particular function (e.g., for presentation as a protein complex, for enhanced immunogenicity, etc.).

Amino acid sequence variants of autoimmune disease associated antigen epitopes and other polypeptides of these compositions can be substitutional, insertional, or deletion variants. A modification in a polypeptide of the invention may affect, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90 or 100 or more non-contiguous or contiguous amino acids of a peptide or polypeptide, as compared to wild-type. A peptide or polypeptide that is associated with an autoimmune response and in particular a pathologic autoimmune response are contemplated for use in methods of the invention.

Deletion variants typically lack one or more residues of the native or wild-type amino acid sequence. Individual residues can be deleted or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of a polypeptide or peptide is affected, such as avidity or affinity for a cellular receptor(s). Non-conservative changes typically involve substituting a residue with one that is chemically dissimilar, such as a polar or charged amino acid for a nonpolar or uncharged amino acid, and vice versa.

Proteins or peptides of the compositions of the invention may be natural, recombinant, or synthesized in vitro. A recombinant protein may be isolated from bacteria or other host cell.

The recombinant proteins or peptides can also be optimized for high level expression in E. coli using codons that are preferred in E. coli. In some embodiments, the invention is directed to engineered antigenic fragments of the autoimmune disease associated autoantigens (nucleic acid and amino acid sequences), which are optimized for expression in E. coli, and may harbor a histidine tag and enterokinase cleavage site to facilitate purification of the protein.

In some embodiments, the codons are optimized for high level expression in E. coli. As used herein, a codon that is “optimized for high level expression in E. coli” refers to a codon that is relatively more abundant in E. coli in comparison with all other codons corresponding to the same amino acid. In some embodiments, at least 40% of the codons are optimized for high level expression in E. coli. In some embodiments, at least 50%, at least 60%, at least 70%, at least 80%, at least 85%, at least 90%, at least 95%, or at least 99% of the codons are optimized for high level expression in E. coli.

It also will be understood that amino acid and nucleic acid sequences may include additional residues, such as additional N- or C-terminal amino acids, or 5′ or 3′ nucleic acid sequences, respectively, and yet still be essentially as set forth in one of the sequences disclosed herein, so long as the sequence meets the criteria set forth above, including the maintenance of biological protein activity (e.g., immunogenicity). The addition of terminal sequences particularly applies to nucleic acid sequences that may, for example, include various non-coding sequences flanking either of the 5′ or 3′ portions of the coding region.

In some embodiments of the compositions of the invention, there is between about 0.001 mg and about 500 mg of total antigen per administration. In some embodiments, about 0.001 mg to about 250 mg, about 0.01 mg to about 100 mg, or about 0.1 mg to about 50 mg is administered. In some embodiments, the amount administered is from about 0.1, about 1, about 10, about 50, or about 100, mg/kg body weight. In one embodiment, an immunologically-effective amount for vaccination against multiple sclerosis is from about 0.1 to about 100 mg/kg.

The present invention contemplates the administration of compositions of the invention to effect a treatment or preventative therapy against the development of a disease or condition associated with autoimmune diseases.

Adjuvants are substances that can be used to specifically augment a specific immune response. In some embodiments, the adjuvants as used herein are Th2 promoting adjuvants. In some embodiments, the adjuvant and the antigen are mixed prior to presentation to the immune system, or presented separately, but into the same site of the subject being immunized. Adjuvants are described by Warren et al. (Ann. Rev. Biochem., 4:369-388, 1986), the entire disclosure of which is hereby incorporated by reference.

In some embodiments, the adjuvant present in the composition is a Th2 promoting adjuvant that increases production of IL-4 in the subject's immune system. In some embodiments, the adjuvant is an aluminum-containing adjuvant. In some embodiments, the adjuvant is selected from the group consisting of AlNa(SO₄)₂, AlNH₄(SO₄), aluminum hydroxide, aluminum phosphate, potassium aluminum sulfate (“alum”) and combinations thereof. In one embodiment, the adjuvant is alum. In some embodiments, the adjuvant, such as alum is administered in an amount ranging from 0.001-0.05 ml/kg. If mice are treated, typically about 100-200 microliters are administered. In some embodiments, the amount of alum administered to a human subject is about 0.05-2.5 ml. In some embodiments, the amount of alum administered to a human subject is about 0.5-2.5 ml. In some embodiments, about 1 ml is administered to a human subject.

In some embodiments, the weight ratio of adjuvant to autoimmune disease associated antigen is from about 1:10 to about 10:1. In some embodiments, the weight ratio of adjuvant to autoimmune disease associated antigen is about 1:9, about 1:8, about 1:7, about 1:6, about 1:5, about 1:4, about 1:3, about 1:2, or about 1:1. In some embodiments, the weight ratio of adjuvant to autoimmune disease associated antigen is about 9:1, about 8:1, about 7:1, about 6:1, about 5:1, about 4:1, about 3:1, or about 2:1.

In some embodiments, the composition further comprises one or more Th2 promoting TLR2 ligands such as Pam3CysSerLys4. In some embodiments the amount of Pam3CysSerLys4 is from about 0.1 μg to about 500 mg. In some embodiments the amount of Pam3CysSerLys4 is from about 1 μg to about 100 mg, from about 10 μg to about 100 mg, from about 50 μg to about 50 mg, from about 100 μg to about 10 mg, or from about 500 μg to about 5 mg. In some embodiments, the amount of Pam3CysSerLys4 is about 50 μg.

In some embodiments, the composition comprises about 0.001-500 mg of antigen; about 0.05-2.5 ml of alum and optionally about 0.1 μg to 500 mg of Pam3CysSerLys4. In some embodiments, the antigen is selected from the group consisting of SEQ ID NOS:1-69. In some embodiments, the antigen is selected from the group consisting of SEQ ID NOS:13-32 and combinations thereof. In one embodiment, the antigen is SEQ ID NO:14.

In some embodiments, the composition comprises about 50-500 mg of antigen; about 0.5-2.5 ml of alum and optionally about 25-250 mg of Pam3CysSerLys4. In some embodiments, the antigen is selected from the group consisting of SEQ ID NOS:1-69. In some embodiments, the antigen is selected from the group consisting of SEQ ID NOS:13-32 and combinations thereof. In one embodiment, the antigen is SEQ ID NO:14.

In some embodiments, the composition comprises about 400 mg of antigen; about 1 ml of alum and optionally about 100 mg of Pam3CysSerLys4. In some embodiments, the antigen is selected from the group consisting of SEQ ID NOS:1-69. In some embodiments, the antigen is selected from the group consisting of SEQ ID NOS:13-32 and combinations thereof. In one embodiment, the antigen is SEQ ID NO:14.

### Pharmaceutical Compositions

As would be understood by one of ordinary skill in the art, when the compositions of the present invention are provided to a subject, it can be in a composition which may contain salts, buffers, or other substances which are desirable for improving the efficacy of the composition.

In some embodiments, the compositions of the invention are formulated as pharmaceutical compositions and administered to a subject. In some embodiments, the compositions are administered as vaccines. In some embodiments, such compositions will generally be dissolved or dispersed in a pharmaceutically acceptable carrier. In some embodiments, the carrier is an aqueous medium.

The term “carrier” denotes an organic or inorganic ingredient, natural or synthetic, with which the active ingredient is combined to facilitate the application of the composition. The characteristics of the carrier depend on the nature of the vaccine and the route of administration. Physiologically and pharmaceutically-acceptable carriers include diluents, fillers, salts, buffers, stabilizers, solubilizers, and other materials. The term “pharmaceutically acceptable” is used to refer to a non-toxic material that is compatible with a biological system such as a cell, cell culture, tissue, or organism.

The compositions can be formulated into liquid preparations for, e.g., nasal, rectal, buccal, vaginal, peroral, intragastric, mucosal, perlinqual, alveolar, gingival, olfactory, or respiratory mucosa administration. Suitable forms for such administration include solutions, suspensions, emulsions, syrups, and elixirs. The compositions can also be formulated for parenteral, subcutaneous, intradermal, intramuscular, intraperitoneal or intravenous administration, injectable administration, sustained release from implants, or administration by eye drops. Suitable forms for such administration include sterile suspensions and emulsions. Such compositions can be in admixture with a suitable carrier, diluent, or excipient such as sterile water, physiological saline, glucose, and the like.

The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Texts, such as Remington: The Science and Practice of Pharmacy, Lippincott Williams & Wilkins; 20th edition (Jun. 1, 2003) and Remington's Pharmaceutical Sciences, Mack Pub. Co.; 18^{th} and 19^{th} editions (December 1985, and June 1990, respectively), incorporated herein by reference in their entirety, can be consulted to prepare suitable preparations. Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

Pharmaceutically acceptable preservatives can be employed to increase the shelf life of the compositions and include, for example, benzyl alcohol, parabens, thimerosal, chlorobutanol and benzalkonium chloride, phenol, sorbic acid, thimerosal, and the like. In some embodiments, a suitable concentration of the preservative can be from 0.02% to 2% based on the total weight although there can be appreciable variation depending upon the agent selected.

In some embodiments, the viscosity of the compositions can be maintained at the selected level using a pharmaceutically acceptable thickening agent. In some embodiments, methylcellulose is used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. In some embodiments, viscous compositions are prepared from solutions by the addition of such thickening agents.

In some embodiments, buffering agents can be employed, such as acetic acid and salts, citric acid and salts, boric acid and salts, and phosphoric acid and salts. In some embodiments of the invention, phosphate buffered saline is used for suspension.

In some embodiments, the compositions are isotonic with the blood or other body fluid of the recipient. In some embodiments, the isotonicity of the compositions can be attained using sodium tartrate, propylene glycol, sugars, sodium chloride, or other inorganic or organic solutes. In some embodiments, sodium chloride is used. Prolonged absorption of the injectable compositions can be brought about by the use in the compositions of agents delaying absorption, for example, aluminum monostearate and gelatin.

In some embodiments, the compositions are administered parenterally. Parenteral vehicles include phosphate buffered saline, sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In some embodiments, the compositions for parenteral administration may be in the form of a sterile injectable preparation, such as a sterile injectable aqueous or nonaqueous solutions, suspensions, and emulsions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Suspensions may be formulated according to methods well known in the art using suitable dispersing or wetting agents and suspending agents. The sterile injectable preparation may also be a sterile injectable solution or suspension in a parenterally acceptable diluent or solvent, such as a solution in 1,3-butanediol. Suitable diluents include, for example, water, Ringer's solution and isotonic sodium chloride solution. In addition, sterile fixed oils may be employed conventionally as a solvent or suspending medium. For this purpose, any bland fixed oil may be employed including synthetic mono- or diglycerides. In addition, fatty acids such as oleic acid may likewise be used in the preparation of injectable preparations.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. Preferably, the form must be sterile and must be fluid to the extent that it may be easily injected. Preferably, it also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants.

In some embodiments, sterile injectable solutions can be prepared by incorporating the autoimmune disease associated antigen and adjuvant in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In the case of sterile powders for the preparation of sterile injectable solutions, in some embodiments, the methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and can be peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include wetting agents, emulsifying and suspending agents, or sweetening, flavoring, or perfuming agents. In some embodiments, the vaccines are provided as liquid suspensions or as freeze-dried products. Suitable liquid preparations include, e.g., isotonic aqueous solutions, suspensions, emulsions, or viscous compositions that are buffered to a selected pH. Transdermal preparations include lotions, gels, sprays, ointments or other suitable techniques. If nasal or respiratory (mucosal) administration is desired (e.g., aerosol inhalation or insufflation), compositions can be in a form and dispensed by a squeeze spray dispenser, pump dispenser or aerosol dispenser. Aerosols are usually under pressure by means of a hydrocarbon. Pump dispensers can preferably dispense a metered dose or a dose having a particular particle size, as discussed below. When in the form of solutions, suspensions and gels, in some embodiments, the formulations contain a major amount of water (preferably purified water) in addition to the active ingredient. Minor amounts of other ingredients such as pH adjusters, emulsifiers, dispersing agents, buffering agents, preservatives, wetting agents, jelling agents, colors, and the like can also be present.

### Therapeutic Methods

The present invention further includes methods for treating or preventing an autoimmune disease or condition.

The compositions can be administered prior, after or both prior to and after the onset of clinical symptoms of the autoimmune disease of interest. In still a further embodiment, the method may include a step that comprises assessing a biological parameter of an autoimmune condition, before and/or after treatment. The methods of the invention may also include assessing a subject's autoimmune status, including the assessment of any autoreactive immune responses.

In some embodiments, the invention provides a method of treating or preventing an autoimmune disease, such as multiple sclerosis, comprising administering to a subject in need thereof an immunologically-effective amount of an autoimmune disease associated antigen or an antigenic fragment or derivative thereof, a Th2 promoting adjuvant and optionally one or more Th2 promoting TLR2 ligands.

In some embodiments, the methods of the invention include treatment of a disease or condition caused by one or more autoimmune disease associated antigens. An autoimmune disease associated antigen and Th2 promoting adjuvant of the invention can be given to induce or modify an immune response in a subject having, suspected of having, or at risk of developing an autoimmune condition or disease, such as multiple sclerosis. Methods may be employed with respect to individuals who have tested positive for autoreactivity or who are deemed to be at risk for developing such a condition or related condition.

In some embodiments, the treatment methods induce production of IL-4. In some embodiments, the treatment methods induce IL-4 and/or cause re-routing of the T-helper cells from the dLNs-CNS route to the mLNs-gut.

In some embodiments, the compositions are administered to mammals, such as mice, to treat EAE. In some embodiments, the methods provide treatment of EAE diseased mice with compositions comprising about 200 μg myelin oligodendrocyte glycoprotein peptide (MOG₃₅₋₅₅) precipitated 1:1 in alum and Pam3CysSerLys4 (50 μg per mouse), administered by i.p. injection. In some embodiments, the treatment induces production by CD4⁺ T cells of IL-4, together with GM-CSF and IL17. Without being bound by theory, the cause of the disease amelioration in EAE is not in the production of IL4 per se, but rather in the re-direction of the CD4⁺ T cells from the dLNs-CNS to the mLNs-gut, without causing overt colitis. As shown in more detail below in the Examples, in relation with reduced disease scores, decreased numbers of infiltrating cells were found in the CNS, the dLNs were reduced, while the mLNs were enlarged and increased numbers of CD4⁺ T cells were found in the mLNs and small intestine lamina propria.

In still further embodiments, the invention includes methods for protecting cells or tissues of a subject from an autoimmune response, particularly a pathogenic autoimmune response, comprising administering to a subject one or more compositions of the invention in an amount sufficient to inhibit the destruction of the cells or tissues comprising the cells, wherein the antigen is associated with the cells and/or tissues.

Typically, compositions of the invention are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner. However, suitable dosage ranges are of the order of ten to several hundreds of nanograms to hundreds of milligrams of autoimmune disease associated antigen per administration. In some embodiments, a single administration is suitable to treat the disease. In other embodiments, subsequent administrations after the initial administration are also contemplated.

In some embodiments, it will be desirable to have multiple administrations of the compositions of the invention, such as about or at least about 3, 4, 5, 6, 7, 8, 9, 10 or more. In some embodiments, the administrations can normally range from 2-3 day to 10 week intervals, or even longer. In some embodiments, the administrations are from one to two week intervals.

In some embodiments, the subject is administered the composition at intervals of 0.5-5 years, for example every two years, to maintain the condition of the immune system.

The compositions and methods of the present invention can also be used in combination with the administration of traditional therapies. These include, but are not limited to, the administration of immunosuppressive or modulating therapies or treatments. In some embodiments, IL-4 is administered in combination with the compositions of the invention. In some embodiments, a retinoid, such as retinoic acid is administered. The present invention also includes methods of inducing an immune response comprising administering to a subject in need thereof an immunologically effective amount of an autoimmune disease associated antigen or an antigenic fragment or derivative thereof, a Th2 promoting adjuvant and optionally one or more Th2 promoting TLR2 ligands to the subject.

In some embodiments, after treatment, the immune cells of the subject maintain the ability to produce Th17 cytokines and are able to combat infections when they arise.

In some embodiments, a combination of antigenic fragments or variants thereof is administered. In other embodiments, the antigenic fragments or variants thereof are administered in more than one composition.

The term “subject” as used herein, refers to animals, such as mammals. For example, mammals contemplated include humans, primates, dogs, cats, sheep, cattle, goats, pigs, horses, chickens, mice, rats, rabbits, guinea pigs, and the like. The terms “subject” and “patient” are used interchangeably.

In some embodiments, the subject is a human. In some embodiments, the subjects are patients who are at high risk of autoimmune disease or who have active autoimmune disease, such as multiple sclerosis.

The administration of the composition may be for either a “prophylactic” or “therapeutic” purpose. When provided prophylactically, the composition is provided in advance of any symptom of autoimmune disease. The prophylactic administration of the composition serves to prevent or attenuate any subsequent autoimmune disease development. When provided therapeutically, the composition is provided upon the detection of a symptom of autoimmune disease. The therapeutic administration of the composition serves to attenuate any actual disease symptoms. In some embodiments, administration of the composition of the invention attenuates multiple sclerosis symptoms in the subject. In some embodiments, administration of the composition of the invention prevents multiple sclerosis in the subject.

The compositions of the invention can be administered to subjects of any age. In some embodiments, the compositions can be administered as a single dose or in a series including one or more additional administrations. In some embodiments, the time interval between the first and second administrations is one week, two weeks, three weeks, 1 month, 2 months, 3 months, 4 months, 5 months, 6 months, 7 months, 8 months, 9 months, 10 months, 11 months, one year, 1.5 years and two years.

In another embodiment, the invention provides a method of treating or preventing an autoimmune disease in a subject by administering high concentration levels of Vitamin A to the subject. In some embodiments, the high levels of Vitamin A are administered orally through the diet. In some embodiments, the high levels of Vitamin A are administered as one or more oral dosage forms in pharmaceutical compositions.

Vitamin A is the name of a group of fat-soluble retinoids, including retinol, retinal, retinoic acid, and retinyl esters. Two forms of vitamin A are available in the human diet: preformed vitamin A (retinol and its esterified form, retinyl ester) and provitamin A carotenoids. Preformed vitamin A is found in foods from various food sources. Beta-carotene is a common provitamin A form. Others include alpha-carotene and beta-cryptoxanthin. Once ingested, they are converted into vitamin A. Both provitamin A and preformed vitamin A are metabolized intracellularly to retinal and retinoic acid, the active forms of vitamin A, to support the vitamin's important biological functions. Both retinyl esters and provitamin A carotenoids are converted to retinol, which is oxidized to retinal and then to retinoic acid. Most of the body's vitamin A is stored in the liver in the form of retinyl esters.

Recommended Dietary Allowances (RDAs) for vitamin A are given as mcg of retinol activity equivalents (RAE) to account for the different bioactivities of retinol and provitamin A carotenoids. Because the body converts all dietary sources of vitamin A into retinol, 1 mcg of physiologically available retinol is equivalent to the following amounts from dietary sources: 1 mcg of retinol, 12 mcg of beta-carotene, and 24 mcg of alpha-carotene or beta-cryptoxanthin. From dietary supplements, the body converts 2 mcg of beta-carotene to 1 mcg of retinol. Currently, vitamin A is listed on food and supplement labels in international units (IUs) even though nutrition scientists rarely use this measure. Conversion rates between mcg RAE and IU are as follows: 1 IU retinol=0.3 mcg RAE; 1 IU beta-carotene from dietary supplements=0.15 mcg RAE; 1 IU beta-carotene from food=0.05 mcg RAE; 1 IU alpha-carotene or beta-cryptoxanthin=0.025 mcg RAE.

In some embodiments, the amount of vitamin A administered in accordance with the invention is at least 3000 mcg RAE per day. In some embodiments, the amount of vitamin A administered is between about 3000-25000 mcg RAE per day, between about 4000-20000 mcg RAE per day, between about 5000-15000 mcg RAE per day, between about 6000-12000 mcg RAE per day, or between about 7000-9000 mcg RAE per day. In some embodiments, the amount of vitamin A administered per day corresponds to at least about 3500 mcg RAE, at least about 4000 mcg RAE, at least about 4500 mcg RAE, at least about 5000 mcg RAE, at least about 5500 mcg RAE, at least about 6000 mcg RAE, at least about 6500 mcg RAE, at least about 7000 mcg RAE, at least about 7500 mcg RAE, at least about 8000 mcg RAE, at least about 8500 mcg RAE, at least about 9000 mcg RAE, at least about 9500 mcg RAE, at least about 10000 mcg RAE, at least about 10500 mcg RAE, at least about 11000 mcg RAE, at least about 11500 mcg RAE, at least about 12000 mcg RAE, at least about 12500 mcg RAE, at least about 13000 mcg RAE, at least about 13500 mcg RAE, at least about 14000 mcg RAE, at least about 14500 mcg RAE, or at least about 15000 mcg RAE. In some embodiments, the amount of vitamin A administered per day corresponds to at least about 7000 mcg RAE. In some embodiments, the Vitamin A is administered in unit dose, spread over 1-5 doses per day.

The length of treatment with high levels of Vitamin A is not limiting. In some embodiments, the subject is treated for a period lasting days, weeks, months or years until the subject experiences an amelioration of disease symptoms. In some embodiments, the subject is treated generally every day from about 0-48 months. In some embodiments, the subject is treated from about 1-52 weeks, from about 4-26 weeks or about 8-32 weeks. Longer periods of treatment are also contemplated by the invention, including treatment for several years or for a continuous period of time. In some embodiments, the subject is administered high Vitamin A daily. In some embodiments, the subject is administered high Vitamin A from 1-7 times per week (i.e., from 1 day to 7 days per week), over a period of about 0-48 months or longer.

In some embodiments, the subject is administered high levels of Vitamin A in combination with the compositions of the invention which comprise an immunologically effective amount of one or more autoimmune disease associated antigens, one or more Th2 promoting adjuvants and optionally one or more Th2 promoting TLR2 ligands. The compositions can be administered to the subject together or separately.

The present invention is further illustrated by the following Examples. These Examples are provided to aid in the understanding of the invention and are not to be construed as a limitation thereof.

### Example 1

Treatment in Th2 conditions following EAE induction in wild type mice prevented progression of EAE by diverting the migration of the CD4⁺ T cells from the dLNs-CNS route to the mLNs-gut.

It was investigated whether the relocation of T-helper cells from the dLNs-CNS route to the mLNs-gut could be induced in EAE wild type mice via the inductions of IL-4 production, by a second immunization using alum adjuvant plus Pam3CSK4. Treatment of EAE wild type mice with MOG₃₅₋₅₅ in Alum, plus Pam3CSK4 (abbreviated MOG/Alum), to increase the Th2 response (MOG/Alum), caused T-helper cells to produce elevated IL-4 levels, without impacting IL-17 and GM-CSF production. Similarly, GATA3 was upregulated with no change in Rorgt (data not shown). Compared to treatment with alum alone, MOG/Alum significantly reduced the EAE clinical scores, caused reduced infiltrations of CD4⁺ T cells in the CNS and increased the percentages of CD4⁺ T cells in the Peyer's patches and SILP, with no overt inflammation in intestinal tissue. Additionally, dLNs were smaller, while the mLNs and Peyer's patches were enlarged, and a4137 and CCR9 were upregulated without downregulation of CCR6, while dendritic cells presented increased Raldh activity. These findings demonstrate that treatment resulting in increased IL-4 production during EAE, without impacting GM-CSF or IL-17 production, caused increased Raldh activity in dendritic cells, imprinting of gut homing markers on CD4⁺ T cells, and reduced EAE severity, thus providing a novel therapeutic avenue for MS.

It is shown herein that immunization under Th2 conditions, viz., by administration of a vaccine composition comprising myelin oligodendrocyte glycoprotein and alum, plus the Tlr2 agonist Pam3CSK4, following EAE induction, causes a dramatic amelioration of EAE in wild type mice. It is demonstrated that Th17 cells produced IL-4, without any impact on IL-17 and GM-CSF. Unexpectedly, the major physiological consequence of IL-4 production during EAE was the diverted migration of the T-helper cells from the draining lymph nodes (dLNS)-CNS route to the mesenteric lymph nodes (mLNs)-gut. The diverted migration of the cells is caused by upregulation of the gut homing receptors CCR9 and integrin a4b7. It is also shown that dendritic cells of the EAE wild type mice treated in Th2 conditions present elevated levels of Radh activity, implicated in retinoic acid production, known to imprint gut homing on T cells. Importantly the treatment, though resulted in gut migration of T cells, did not cause overt colitis.

Importantly, this treatment follows EAE induction, rather than preceding disease induction.

Another treatment previously believed to protect from EAE by induction of Th2 response was tested (Falcone et al., 1995; Forsthuber et al. 1995), namely immunization with MOG₃₅₋₅₅ in incomplete Freund's adjuvant (MOG/IFA). However different from the previous studies, mice were treated with MOG₃₅₋₅₅ in incomplete Freund's adjuvant, 7 days after EAE was induced, similarly to what was done with MOG₃₅₋₅₅ in Alum, plus Pam3CSK4, discussed above. Treatment of EAE mice with MOG₃₅₋₅₅ in incomplete Freund's adjuvant (MOG/IFA), did not induce production of IL-4, but rather reduced production of IL-17 and Gm-CSF. Additionally, this treatment did not cause re-routing of the T-helper cells from the dLNs-CNS route to the mLNs-gut.

The Following Materials and Methods were used in the Examples provided herein:

### Mice

All experiments were conducted on 8-16 week old mice. All mouse procedures were approved by the AMC Institutional Animal Care and Use Committee.

### EAE Induction

To induce EAE, emulsions containing 4 mg/ml of Mycobacterium Tuberculosis in Incomplete Fruend's Adjuvant (Sigma) and 2 mg/ml MOG₃₅₋₅₅ peptide were injected subcutaneous above each hind flank of the mouse. Additionally, 3 μg/ml of pertussis toxin (List Biological Inc.) were injected i.p. on day 0 and day 1. Beginning on day 7, disease progression was monitored. Clinical scoring was established as follows: Score 1: flaccid tail, Score 2: Weak hind limbs, Score 3: Hind limb paralysis, Score 4: Quadriplegia. Female mice were used in all EAE experiments and disease was induced at 8-10 weeks of age.

### MOG/Alum Immunization

EAE was induced in 9-10 weeks old females. Seven days post induction, mice were immunized i.p. with MOG/Alum (2 mg/ml) or Alum alone. Mice injected with MOG/Alum also received two 50 μg i.p. injections of Pam3CSK4 (Invivogen), a stimulator of Th2 response.

### Histology

Upon euthanasia brain and spinal cord were fixed in 10% paraformaldehyde, paraffin embedded and sections were cut and stained with hematoxylin and eosin (H&E). Cross sections and stainings were performed by Mass Histology Service, Inc. Microscopic examination of the sections was performed using an Olympus BX51 instrument (Olympus).

### Staining for FACS Analysis

Intranuclear staining followed surface staining and was conducted using fixation and permeabilization buffers (ebioscience) with Protocol B. Flow cytometry analysis of transcription factors in T lymphocytes. Methods Mol Biol 647, 377-390 (2010)).

### Intracellular Cytokine Staining

Cells were stimulated for six hours with 50 ng/ml PMA and 500 ng/ml Ionomycin in CM at 37° C., with 10 μg/ml Brefeldin A. Cells were stained for surface markers, followed by fixation in 4% paraformaldehyde. Permeabilization of cells with 0.05% saponin buffer was performed before intracellular staining.

### Aldeflour Assay

To measure aldehyde dehydrogenase activity, we used an aldeflour assay kit from Stem Cell Technologies Inc. Cells were blocked and then kept in aldeflour assay buffer throughout experiment. 0.5 ml aldeflour substrate was added to 1×10⁶ cells in 200 ml assay buffer in the presence or absence of 0.5 ml DEAB inhibitor. All cells incubated at 37° C. for 30 min, followed by staining for surface markers at 4° C. for 15 min and FACS analysis.

### Flow Cytometry

Flow cytometry analyses were performed on a an LSR or on FacsCalibur upgraded at three lasers and 8 colors (Cytek). Data was analyzed using FlowJo software (Tree Star Inc.)

### Statistical Analysis

Differences between MOG/Alum-treated versus Alum-treated EAE mice were determined by a two-tailed Student t test (unequal variance). P≦0.05 was considered significant. All values were expressed as mean±Standard error (s.e.m.).

1. A composition comprising an immunologically-effective amount of one or more autoimmune disease associated antigens or antigenic fragments or derivatives thereof and one or more Th2 promoting adjuvants and optionally one or more Th2 promoting TLR2 ligands.

2. The composition of claim 1 further comprising a pharmaceutically acceptable carrier.

3. The composition of claim 1, wherein the one or more autoimmune disease associated antigens or antigenic fragments or derivatives thereof is associated with multiple sclerosis.

4. The composition of claim 3, wherein the one or more autoimmune disease associated antigens or antigenic fragments or derivatives thereof is selected from the group consisting of myelin basic protein, myelin associated glycoprotein, alphaB-crystallin, S100beta, proteolipid protein (PLP) and myelin oligodendrocyte glycoprotein (MOG).

5. The composition of claim 4, wherein the one or more autoimmune disease associated antigens is selected from the group consisting of MOG₃₅₋₅₅ mouse fragment, MEVGWYRSPFSRVVHLYRNGK (SEQ ID NO:13); MOG human fragment, MEVGWYRPPFSRVVHLYRNGK (SEQ ID NO:14); MAG₂₈₇₋₂₉₅ human fragment, SLLLELEEV (SEQ ID NO:15); MAG₂₈₇₋₂₉₅ mouse fragment, SLYLDLEEV (SEQ ID NO:16); MAG₅₀₉₋₅₁₇ mouse and human fragment, LMWAKIGPV (SEQ ID NO:17); MAG₅₅₆₋₅₆₄, human fragment, VLFSSDFRI (SEQ ID NO:18); MAG₅₅₆₋₅₆₄, mouse fragment, VLYSPEFRI (SEQ ID NO:19); MBP human fragment, SLSRFSWGA (SEQ ID NO:20); MBP mouse fragment, SLSRFSWGG (SEQ ID NO:21); MOG mouse and human fragment, KVEDPFYWV (SEQ ID NO:22); MOG mouse and human fragment, RTFDPHFLRV (SEQ ID NO:23); MOG mouse and human fragment, FLRVPCWKI (SEQ ID NO:24); MOG mouse and human fragment, KITLFVIVPV (SEQ ID NO:25); MOG mouse and human fragment, VLGPLVALI (SEQ ID NO:26); MOG mouse and human fragment, TLFVIVPVL (SEQ ID NO:27); MOG mouse and human fragment, RLAGQFLEEL (SEQ ID NO:28); PLP₈₀₋₈₈ mouse and human fragment, FLYGALLLA (SEQ ID NO:29); and combinations thereof.

6. The composition of claim 1, wherein the one or more autoimmune disease associated antigens or antigenic fragments or derivatives thereof is present in the composition at about a 1:1 ratio by weight with the adjuvant.

7. The composition of claim 1, wherein the adjuvant increases production of IL-4 upon administration in a subject and causes re-routing of the harming immune cells to places where they can be of no harm.

8. The composition of claim 1, wherein the adjuvant is an aluminum containing adjuvant.

9. The composition of claim 1, wherein the adjuvant is selected from the group consisting of AlNa(SO₄)₂, AlNH₄(SO₄), aluminum hydroxide, aluminum phosphate, potassium aluminum sulfate (“alum”), and combinations thereof.

10. The composition of claim 1, wherein the Th2 promoting TLR2 ligand is selected from the group consisting of Pam3CysSerLys4 (Pam3CSK4), 3-palmytoil-s-glycerylcysteine, Pam2CSK4, diacetylated lipopetide FSL-1 (Pam2CGDPKHPKSF), lipoteichoic acid, peptidoglycan and combinations thereof.

11. A method of treating or preventing an autoimmune disease comprising administering to a subject in need thereof an immunologically-effective amount of one or more autoimmune disease associated antigens or antigenic fragments or derivatives thereof, one or more Th2 promoting adjuvants and optionally one or more Th2 promoting TLR2 ligands.

12. The method of claim 11, wherein the autoimmune disease is multiple sclerosis.

13. The method of claim 12, wherein the one or more autoimmune disease associated antigens or antigenic fragments or derivatives thereof is selected from the group consisting of myelin basic protein, myelin associated glycoprotein, alphaB-crystallin, S100beta, proteolipid protein (PLP) and myelin oligodendrocyte glycoprotein (MOG).

14. The method of claim 13, wherein the one or more autoimmune disease associated antigens is selected from the group consisting of MOG₃₅₋₅₅ mouse fragment, MEVGWYRSPFSRVVHLYRNGK (SEQ ID NO:13); MOG human fragment, MEVGWYRPPFSRVVHLYRNGK (SEQ ID NO:14); MAG₂₈₇₋₂₉₅ human fragment, SLLLELEEV (SEQ ID NO:15); MAG₂₈₇₋₂₉₅ mouse fragment, SLYLDLEEV (SEQ ID NO:16); MAG₅₀₉₋₅₁₇ mouse and human fragment, LMWAKIGPV (SEQ ID NO:17); MAG₅₅₆₋₅₆₄, human fragment, VLFSSDFRI (SEQ ID NO:18); MAG₅₅₆₋₅₆₄, mouse fragment, VLYSPEFRI (SEQ ID NO:19); MBP human fragment, SLSRFSWGA (SEQ ID NO:20); MBP mouse fragment, SLSRFSWGG (SEQ ID NO:21); MOG mouse and human fragment, KVEDPFYWV (SEQ ID NO:22); MOG mouse and human fragment, RTFDPHFLRV (SEQ ID NO:23); MOG mouse and human fragment, FLRVPCWKI (SEQ ID NO:24); MOG mouse and human fragment, KITLFVIVPV (SEQ ID NO:25); MOG mouse and human fragment, VLGPLVALI (SEQ ID NO:26); MOG mouse and human fragment, TLFVIVPVL (SEQ ID NO:27); MOG mouse and human fragment, RLAGQFLEEL (SEQ ID NO:28); PLP₈₀₋₈₈ mouse and human fragment, FLYGALLLA (SEQ ID NO:29); and combinations thereof.

15. The method of claim 11, wherein the one or more autoimmune disease associated antigens or antigenic fragments or derivatives thereof is present in the composition at about a 1:1 ratio by weight with the adjuvant.

16. The method of claim 11, wherein the adjuvant increases production of IL-4 upon administration in a subject and causes re-routing of the harming immune cells to places where they can be of no harm.

17. The method of claim 11, wherein the adjuvant is an aluminum containing adjuvant.

18. The method of claim 11, wherein the adjuvant is selected from the group consisting of AlNa(SO₄)₂, AlNH₄(SO₄), aluminum hydroxide, aluminum phosphate, potassium aluminum sulfate (“alum”), and combinations thereof.

19. The method of claim 11, wherein the Th2 promoting TLR2 ligand is selected from the group consisting of Pam3CysSerLys4 (Pam3CSK4), 3-palmytoil-s-glycerylcysteine, Pam2CSK4, diacetylated lipopetide FSL-1 (Pam2CGDPKHPKSF), lipoteichoic acid, peptidoglycan and combinations thereof.
